(19) United States
(12) Patent Application Publication
(10) Pub. No.: US 2017/0348178 A1
(43) Pub. Date: Dec. 7, 2017

(54) Automated Fundal Massager

(71) Applicant: Tamy DeLeon, Waimanalo, HI (US)

(72) Inventor: Tamy DeLeon, Waimanalo, HI (US)

(21) Appl. No.: 15/615,142

(22) Filed: Jun. 6, 2017

Related U.S. Application Data
(60) Provisional application No. 62/346,346, filed on Jun. 6, 2016.

(51) Int. Cl. A61H 7/00; A61B 17/12; A61H 11/02; A61H 11/00

(52) U.S. Cl. CPC A61H 7/004; A61H 11/02; A61B 17/12; A61B 2017/12004; A61H 2011/005

(57) ABSTRACT

An automated fundal massager for massaging the uterus in a patient, the apparatus comprising at least one attachment mechanism securing the massager to the patient, a body housing the massager and at least one massager pressingly engaging the fundus having at least one power supply, at least one fundus engagement device, at least one fundus engagement device cover, and at least one system controller in communication with the fundus engagement device, whereby a massager is placed on the patient to engage the fundus of the uterus through massage postpartum to maintain uterine contractility thereby managing postpartum bleeding.

### CROSS-REFERENCE TO RELATED APPLICATIONS

This application claims priority of provisional Patent Application No. 62/346,346 filed Jun. 6, 2016.

### FIELD OF THE INVENTION

The invention generally relates to an apparatus that is placed on the lower part of a birth mother's abdomen following the birth to provide direct pressure to massage the fundus of the uterus (top portion of uterus). In particular, the invention relates to an apparatus for massaging the fundus of the uterus to maintain uterine contractility to prevent excessive postpartum bleeding and hemorrhage and allow a nurse that would normally perform this function to be free to attend to other critical tasks.

### BACKGROUND

According to the World Health Organization (2012), postpartum hemorrhage (PPH) is the leading cause of maternal mortality worldwide with a prevalence of approximately 6%. The most common cause of PPH is uterine atony or uterine flaccidity. Active management of the third stage of labor or the period immediately following the birth of the baby to delivery of placenta and the immediate postpartum period by a skilled attendant such as a nurse is vital to help decrease mortality and morbidity of the mother. The World Health Organization (2012) stated that health systems face enormous constraints that hinder the delivery of immediate and emergent obstetrical care, which is vital to saving the lives of women who develop PPH.

A nurse or skilled attendant present at the birth is required to assess and actively manage maternal bleeding and fundal or uterine firmness using fundal massage in the immediate postpartum period following the birth of a fetus. Uterine contractility or firmness of the uterus is vital to help control and maintain uterine bleeding. Fundal massage is the initial, most effective, noninvasive action used to attain and maintain uterine contractility and firmness. The nurse or skilled attendant has a standard of practice that mandates they perform a manual fundal massage using the fingertips of one or both hands at the lower half of the abdomen on the fundus or top of the descending uterus using circular motions in order initiate and maintain uterine contractility. Fundal massage immediately after birth is often dependent upon bleeding and the firmness of the uterus. If the uterus maintains a soften state or readily softens, the nurse is required to continually perform fundal massage to keep the fundus firm. Frequently, the bedside nurse is pulled away to attend to other urgent or emergent needs such as assisting in resuscitation of the newborn leaving the mother at risk for developing a PPH.

### SUMMARY OF THE INVENTION

The present invention overcomes these shortcomings by providing an automated fundal massager that is placed and secured to the birth mother's body with the massager side down, engaging the abdomen at the fundal point, just below the umbilicus to apply direct pressure and massage the top of the uterus or fundus after birth thereby initiating and maintaining uterine contractility and fundal firmness thus preventing excessive postpartum bleeding and potential PPH. The apparatus may consist of a attachment method for securing the fundal massager to the body in the desired location, a massager to engage the fundus and system controls that determine the level of the massage. The automated fundal massager allows the birth mother to receive the treatment immediately after birth to prevent excessive postpartum bleeding and potential PPH while freeing medical personnel to perform other critical duties.

There have thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. Additional benefits and advantages of the present invention will become apparent to those skilled in the art to which the present invention relates from the subsequent description of the preferred embodiment and the appended claims, taken in conjunction with the accompanying drawings. It is important, therefore, that the claims be regarded as including such equivalent constructions insofar as they do not depart from the spirit and scope of the present invention,

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### DESCRIPTION OF THE DRAWINGS

FIG. 1 is a pictorial drawing of the fundal massager attached to a birth mother.

FIG. 2 is front view of the fundal massager.

FIG. 3 is a rear view of a fundal massager.

FIG. 4 is top view of a fundal massager,

FIG. 5 is top view of a fundal massager with a pneumatic pressure applicator.

FIG. 6 is a view of a charging station for multiple fundal massagers.

FIG. 7 is a view of a storage/carrying case for the fundal massager.

### DETAILED DESCRIPTION OF THE INVENTION

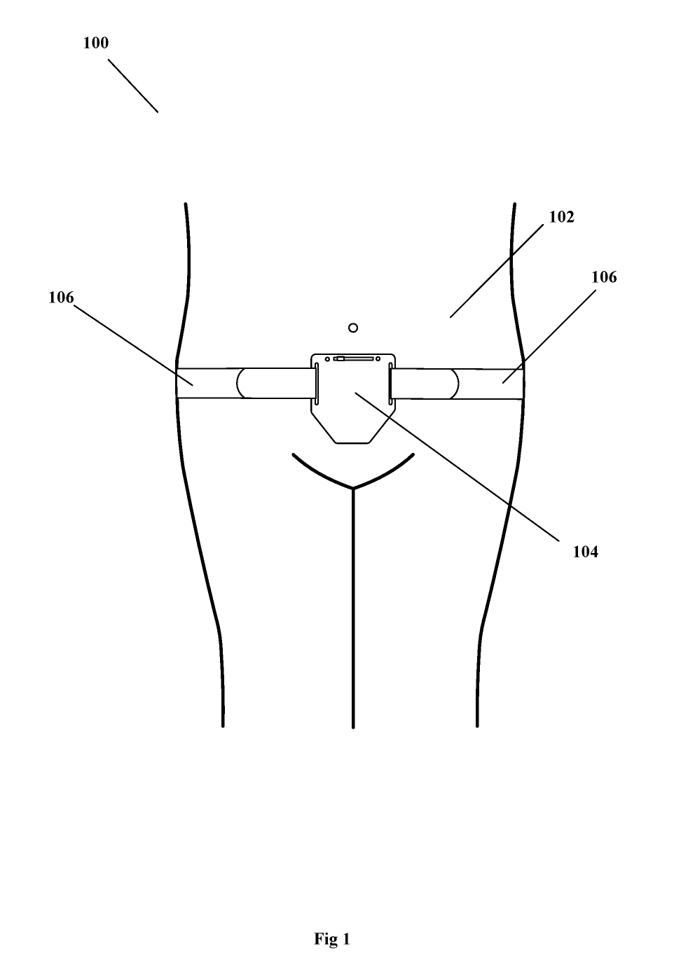

FIG. 1 is a illustrative view 100 that shows the fundal massager 104 attached to a birth mother 102 using housing 106 to secure the fundal massager 104 in the desired location. The fundal massager 104 is placed on the abdomen above the fundus of the uterus and the straps 106 are tightened sufficiently to prevent the movement of the fundal massager 104 while not injuring the birth mother 102. Nurses or other medical personnel may check the positioning and securing of the fundal massager 104 so that the fundal massager 104 will provide direct pressure to the fundus of the uterus thereby controlling excessive bleeding or postpartum hemorrhaging. The positioning of the fundal massager 104 may be based on each individual birth mother's 102 anatomy.

A single strap 106 or multiple straps may be used to secure the fundal massager 104 to the birth mother 102. In the preferred embodiment, a single strap 106 may be used with one end of the strap 106 entering one side of the fundal massager 104 through the strap attachment point 204 and folded back on to itself and secured by a fastener which may be selected from the following, including but not limited to, snaps, buckle, ratchets, quick-connects, friction, snaps, hooks, and hooks and loops. The remainder of the of the strap 106 may be placed behind the birth mother 102 around her back then the other end on the strap 106 may enter the other side of the fundal massager 104 through the strap attachment point 204 and secured in the same fashion as described above. Before securing the strap 106 in place, the strap 106 is tightened sufficiently securing the strap 106 in place thereby maintaining the placement of the fundal massager 104. In the preferred embodiment, hooks and loops are the preferred method of securing and fastening the strap 106 to the birth mother 102. Other embodiments, may include but are not limited, to these below described embodiments, one side of the strap 106 being permanently affixed to the fundal massager 104 at the strap attachment point 204, the straps 106 ends coming through the strap attachment points 204 and fastening to each other on the top of the fundal massager 104. Also, the strap 106 that is used for the fetal monitor may be used to secure the fundal massager 104.

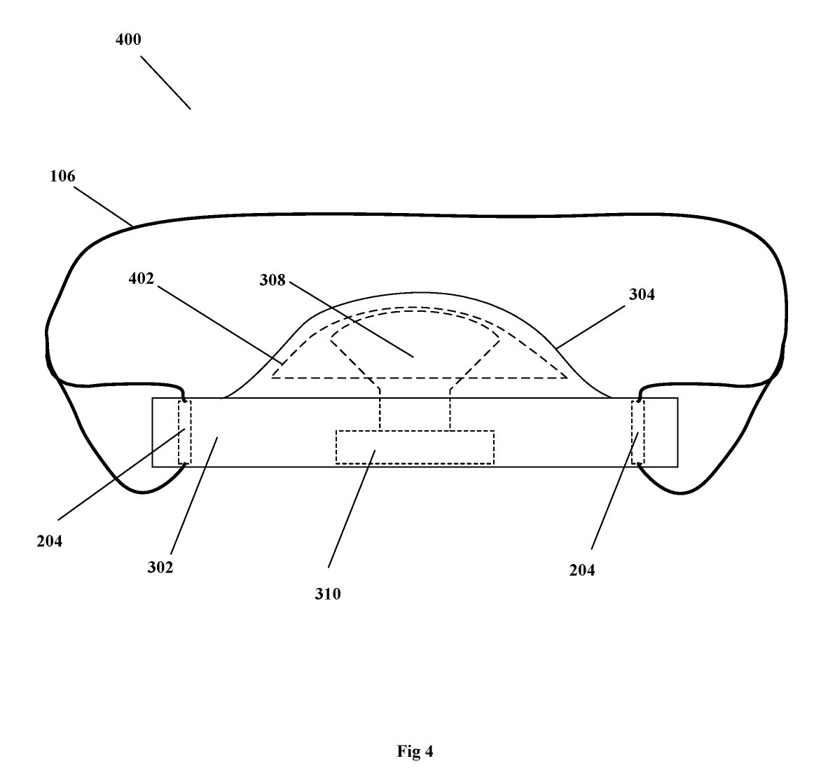

The straps 106 may be flexible or non-flexible. A non-flexible strap 106 may be desired for birth mothers 102 that need a deep and firm massage of the fundus to prevent the bleeding or PPH and the birth mother 102 can tolerate the additional discomfort due to the inflexibility whereas a flexible strap 106 may be desired for birth mothers 102 that have a lower tolerance of discomfort and the need for a deep massage may be lessened. The straps 106 may be constructed from materials known to one skilled in the art that would be appropriate for use in medical facilities. Additionally, different widths of straps 106 may be used to accommodate different size of birth mothers 102. A wider strap 106 for a larger mother may help in maintaining the desired position and prevent the fundal massager 104 from flipping while it is in operation due to the birth mother 102 size. The straps 106 attachment to the fundal massager 104 are further illustrated in FIG. 4.

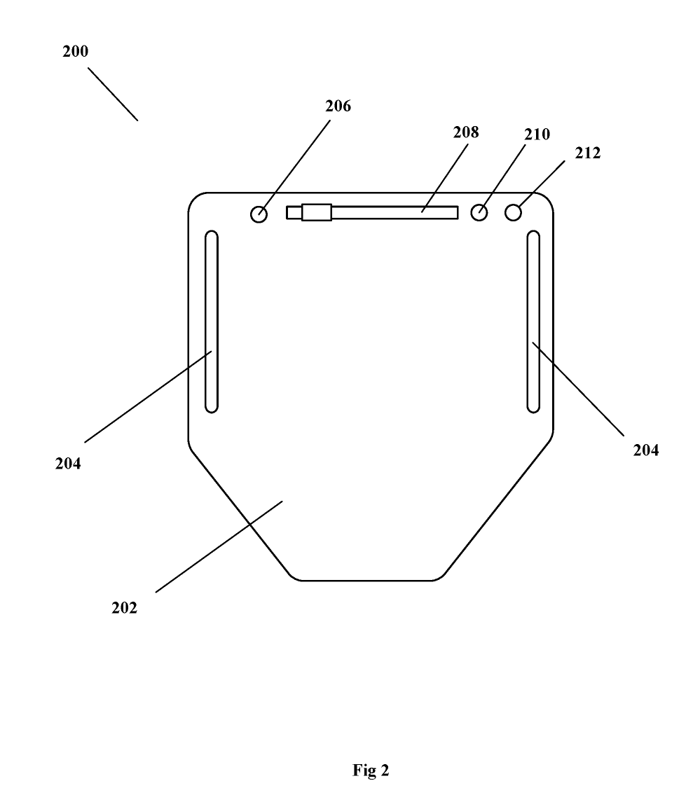

FIG. 2 is a front view 200 of the fundal massager 104. This view 200 of the fundal massager 104 shows a housing cover 202, strap attachment points 204, a power control 206, a speed control 208, a mode control 210, and a frequency control 212. The housing cover 202 covers and protects the fundal massager's 104 internal components described later from the harsh environment of a delivery room and prevents contamination. The housing cover 202 may be constructed from materials suitable for a medical facility environment and should be cleanable and sanitizeable. Additionally, the housing cover 202 may contain a seal, such that when the housing cover 202 and housing body 302 are attached so that no material may enter the fundal massager 104, more specifically, no liquid material such as blood may enter so to prevent person to person contamination. The strap attachment points 204 allow the strap or straps 106 to be attached to the fundal massager 104 and secured around the birth mother's 102 body and placed in the desired position as described above.

The power control 206 turns the fundal massager 104 on and off. In the preferred embodiment, the power control 206 may be a push button switch to easily turn on the fundal massager 104 but also may be selected from other switches known to one skilled in the art, including but not limited to, slide switches and rotary switches. The speed control 208 may be used to select the speed at which massager roller 308 articulates to massage the fundus. In a preferred embodiment, the speed control 208 is a slide switch where the left most position provides no movement and as the slide is positioned further to the right from the left most starting point the speed increases proportionally. Other types of speed control devices known to one skilled in the art may be used to control and set the speed, including but not limited to a rotary switch. Additionally, the speed control 208 may also use discreet speed settings instead of the proportional setting of a slide switch where the medical personnel may select a specific setting based on the contractility of the fundus. In addition to the power control 206 and the speed control 208, the system may also have a mode control 210 that allows the medical personnel to select differing modes based on the type of fundal massage required for a specific birth mother 102. The different modes may include but are not limited to a continuous mode, a ramping mode, and a pulse mode. In the continuous mode, medical personnel may set the different controls to the desired settings and start the fundal massager 104 and the fundal massager 104 would continue massaging until the medical personnel stopped the fundal massager 104. In the ramping mode, medical personnel may set the controls specifically the speed control 208 to the desired maximum level, and start the fundal massager 104. The fundal massager 104 would start at the lowest speed and may increase the speed up to the maximum speed set over a specific desired time then stop and start again at the lowest speed and continue the process of ramping the speed until medical personal stop the fundal massager 104. The pulse mode allows fundal massager 104 to periodically turn on and provide the massaging action to the fundus at the desired speed and interval then turn off. The frequency control 212 may be used to control how often the massager may turn on and turn off during a specific period of time. This mode may allow the birth mother 102 some period of rest between the massage events.

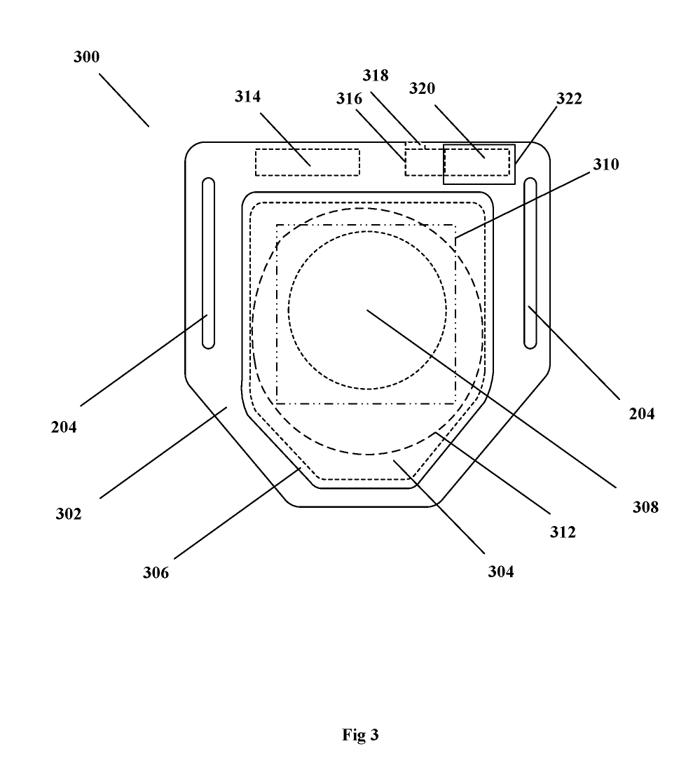

FIG. 3 is a rear view 300 of a fundal massager 104. This view 300 illustrates a fundal massager 104 that may consist of a body housing 302, a roller cover 304, a cover attachment 306 and strap attachment points 204. Inside the body housing 302, the fundal massager 104 may further consist of a motor 310, a system controller 314, a power supply 316, a power connection 318, and a battery 320. In the preferred embodiment, the body housing 302 and the housing cover 202 are fashioned in a trapezoid design to better accommodate the birth mother's 102 abdominal area but other shapes may be used by one skilled in the art.

In the preferred embodiment, the roller 308 may be affixed to the motor 310 wherein the motor 310 may move the roller 308 within the defined space of the roller area 312 to provide a massage to the fundus similar to that of a nurse or other medical personnel after the delivery. The roller cover 304 is attached to the body housing 302 via a cover attachment 306. In the preferred embodiment, the roller cover 304 is semi-permanently affixed to the body housing 302 by physically stitching or other methods known to one skilled in the art for adhering the roller cover 304 to the body housing 302. In another embodiment, the roller cover 304 may be removably attached to the body housing 302 by hooks and loops or other fastening methods for making the material removable known to one skilled in the art. The roller cover 304 may be selected from silicone, pleather or other material suitable for medical use. The roller 308 that is affixed to the motor 310 may be controlled by the system controller 314 to implement the type of massage selected by the medical personnel.

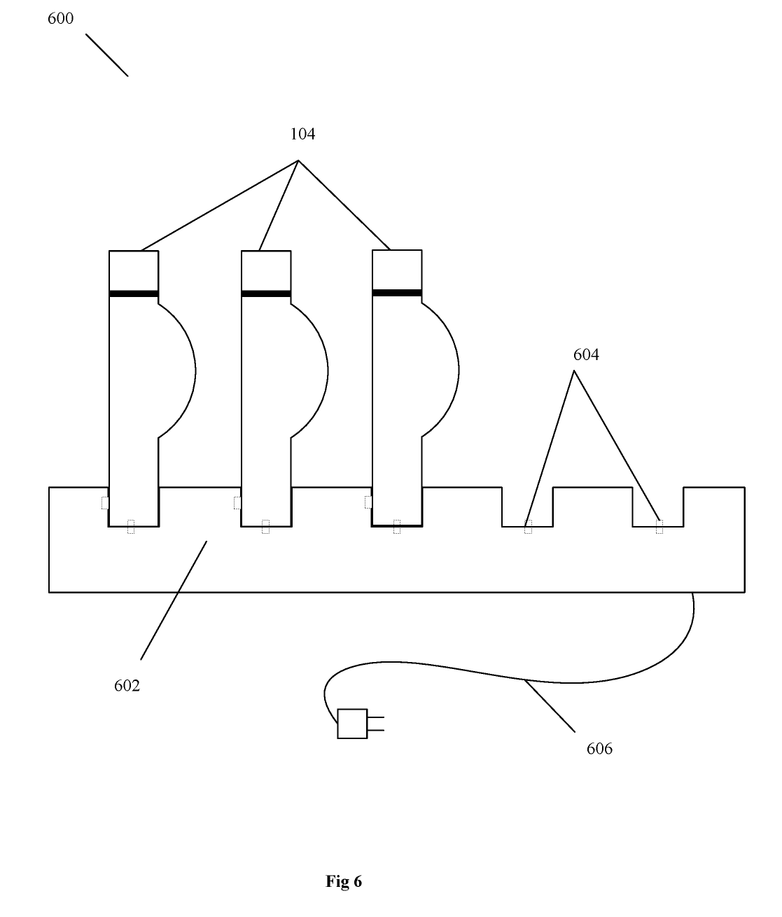

The system controller 314 and the motor 310 maybe supplied power by a power supply 316. The power supply 316 may consist of a power connection 318, a battery 320, a battery cover 322, and power conversion. The power connection 318, may supply either 120 volts AC, 220 volts AC or DC voltage direct current to the fundal massager 104. In the preferred embodiment, the motor 310 and system controller 314 operate on DC. In addition to the external power supplied, an internal battery 320 may be used to power the motor 310 and system controller 314 through the power supply 316. The battery 320 may be removable and replaced through a battery cover 322 or it may be rechargeable either through a charging station described in FIG. 6 or through power connection 318 and the power supply 316. The power supply 316 provides power to the system controller 314 and the motor 310 through a power control switch 206.

The system controller 314 may take inputs from the controls described in FIG. 2 such as a power control 206, a speed control 208, a mode control 210 and a frequency control 212. From these inputs, the system controller 314 may manipulate the motor 310 and roller 308 to perform the desired massage to the fundus. The preferred embodiment uses analogue controls to select the massaging technique desired by the medical personnel. In another embodiment, these analog controls may be replaced with digital controls to perform the same functions. Additionally, a microcontroller, programmable logic unit or other programmable unit may be used to perform the function of the system controller 314 and analog inputs. The system controller 314 may be controlled remotely by wired and wireless communication.

FIG. 4 is a top view 400 of a fundal massager 104. This view of the fundal massager 104 shows a roller cushion 402 that covers the roller 308, straps 106, the body housing 302, the roller cover 304. This view further illustrates the roller 308 protruding from the body housing 302 and connected to the motor 310. This view also shows that the roller 308 resides under the roller cover 304, wherein the roller 308, through the motor 310, moves around inside the covering, providing the massage. The roller cushion 402 may provide some cushioning between the roller 308 and the birthing mother to prevent bruising during operation. The cushion may be affixed to the roller 308 or the roller cover 304.

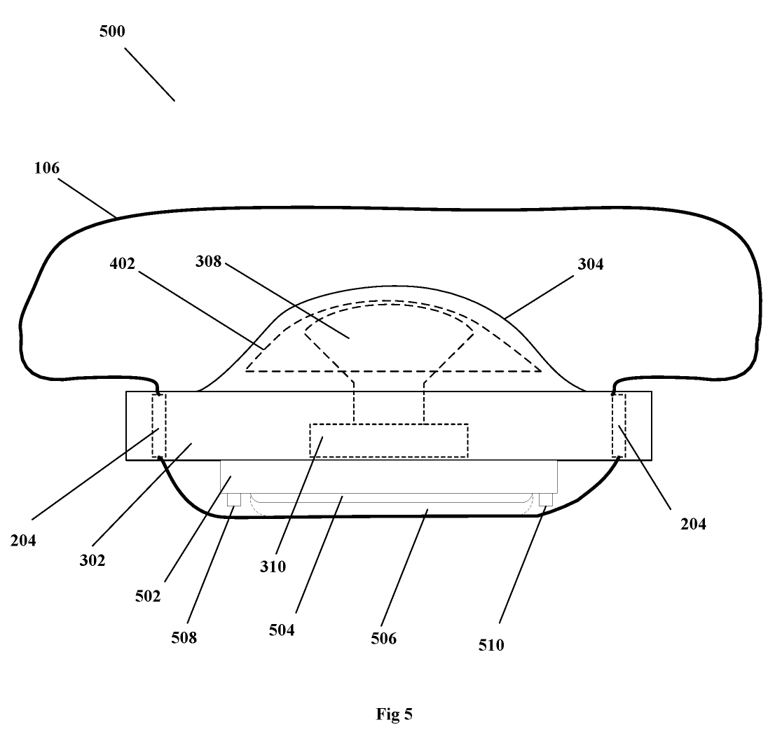

FIG. 5 is a top view of a fundal massager 104 with a pneumatic pressure applicator 500. The pneumatic pressure applicator 500 may consist of a pneumatic housing 502, a pneumatic bladder 504, a pump control 508 and a pump release 510. The fundal massager 104 is designed to provide a deep massage of the fundus of the uterine to help control and prevent bleeding and possibly aver PPH. The straps 106 may be tightened, which secures the fundal massager 104 to the birthmother's body to hold the fundal massager 104 in the desired position and to provide pressure to be fundus of the uterus. Additionally, some birth mothers 102 may need additional pressure applied to the fundus of the uterus to prevent bleeding and PPH. To increase the pressure, a pneumatic pressure applicator 500 may be placed on the back of the fundal massager 104 or may be an integral part of the fundal massager 104. When air is pumped into the pneumatic bladder 504 via the pump control 508, the expanded pneumatic bladder 506 may provide additional pressure to the fundus of the uterus. Once the massaging has been completed, then medical personnel may press the pump release 510 to release the pressure to allow the fundal massager 104 to be more easily removed from the birthing mother. The amount of pumping by pump control 508 may determine the additional of pressure applied to fundus.

FIG. 6 is a view of a charging station 600 for multiple fundal massagers 104. The charging station 600 may consist of a battery charger 602, a massager power connection 604 and a charging power connection 606. This view illustrates three fundal massagers 104 charging in a five-slot charging station 600. The charging station 600 in the current embodiment allows for up to five fundal massagers 104 to be charged simultaneously. The number of fundal massagers 104 being charged could be increased or decreased based on the requirements of the medical facility. For example, hospitals that only do a few deliveries may want a smaller charging station but other hospitals that have much larger birthing centers may want multiple charging stations 600 or a single charging station 600 with multiple positions for multiple fundal massagers 104. In the preferred embodiment, the power connection 606 is connected to a standard wall socket that provides 120 volts AC. This power is then supplied to the battery charger 602 within the charging station 600. This charging power is then supplied to the fundal massagers 104 by the massager power connection 604 thus charging the internal battery 320. This power is then supplied to the fundal massager 104 to charge the battery 320. Upon completing the charging of the fundal massager 104 the charging station 600 may provide some type of indication to the medical personnel when a fundal massager 104 is fully charged. Additionally, the charging station 600 may provide indication that a specific fundal massager 104 is charging.

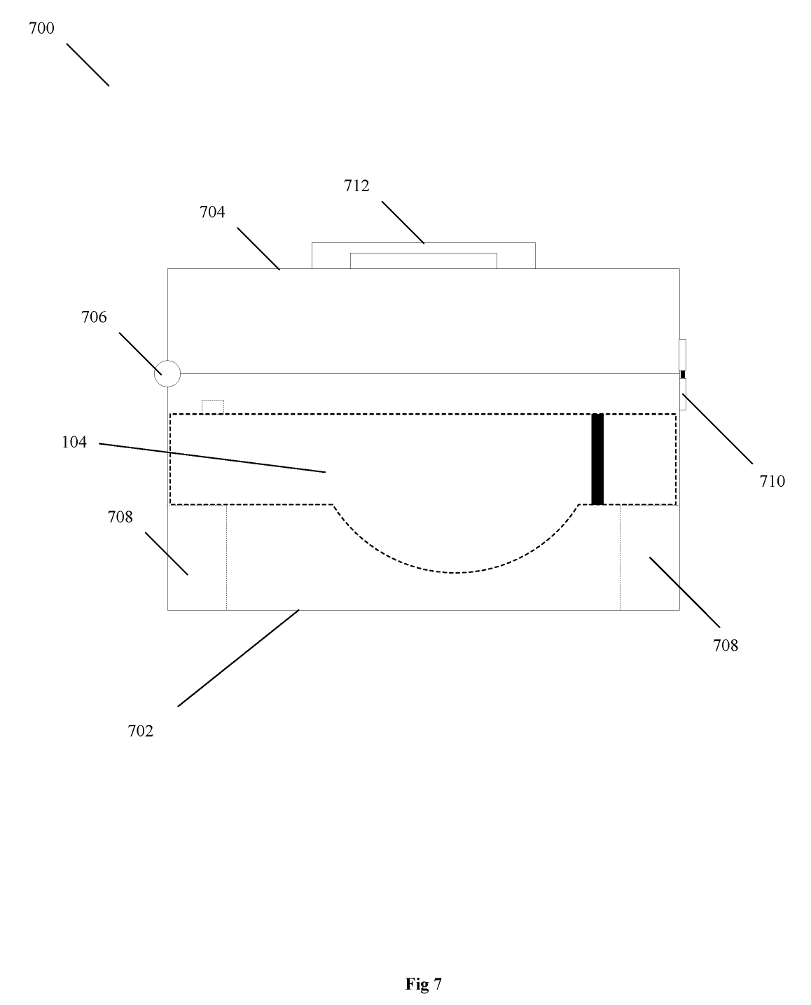

FIG. 7 is a view of a storage/carrying case 700 for the fundal massager 104. This view illustrates a container 702 with a fundal massager 104 inside with the lid 704 in the closed position. The case 700 may consist of a container 702, a lid 704, a hinge 706, positioners 708, a lock 710, and handle 712. The container 702 may have positioners 708 in each corner to suspend the fundal massager 104. The fundal massager 104 is placed massager side down where such the positioner 708 engages the corners and suspends the fundal massager 104 such that the roller 308 and the roller cover 304 do not touch the bottom of the storage case 702. A pad may be placed inside the lid 704 to provide pressure to the fundal massager 104 to maintain the fundal massager 104's position while it is being stored or transported. Additional padding may be added to prevent the fundal massager 104 from being damaged while in storage and during transport. Another embodiment allows a strap inside the container 702 to secure the fundal massager 104 in place. The strap may use hooks and loops or other known securing methods known to one skilled in the art to secure the fundal massager 104.

In the preferred embodiment, the lid 704 is hingedly attached by a hinge 706 to the container 702 allowing all the parts of the fundal massager case 700 to remain together. Other embodiments may allow the lid 704 to separate from the container 702 or the lid 704 may be attached to the container 702 by straps. Additionally, the lid 704 may be secured in place by a lock 710 or by other means known by one skilled in the art including but not limited to straps with hooks and loops.

In this preferred embodiment, the ease 700 may only carry a single fundal massager 104. Other embodiments may be designed to transport multiple fundal massagers 104 in a single case 700. Additionally, the case 700 may be designed such that the fundal massager 104 may be charged while it is inside the case 700.

Having thus described the invention, I claim:

1. An automated fundal massager for massaging the uterus in a patient, the apparatus comprising:
a. at least one attachment mechanism securing the massager to the mother having—
i. at least one fastener;
b. a body housing the massager; and
c. at least one massager pressingly engaging the fundus having—
i. i. at least one power supply,
ii. ii. at least one fundus engagement device,
iii. iii. at least one fundus engagement device cover, and
iv. at least one system controller in communication with the fundus engagement device,
whereby a massager is placed on the patient to engage the fundus of the uterus through massage to maintain uterine contractility thereby managing postpartum bleeding.

2. The massager of claim 1, where the attachment mechanism is selected from flexible or non-flexible straps.

3. The massager of claim 1, where the fastener is selected from snaps, buckle, ratchets, quick-connects, friction, snaps, hooks, and hooks and loops.

4. The massager of claim 1, where the power supply is selected for alternating current (AC) or direct current (DC).

5. The massager of claim 4, where the power supply is selected for 120 volts AC and 220 Volts.

6. The massager of claim 1, where the power supply is rechargeable.

7. The massager of claim 6, where the power supply is removable for charging.

8. The massager of claim 7, where multiple power supplies may be charged simultaneously.

9. The massager of claim 1, where the fundus engagement device cover silicone, pleather, plastic, latex, and cloth.

10. The massager of claim 1, where the fundus engagement device cover is removable

11. The massager of claim 10, where the fundus engagement device cover is disposable.

12. The massager of claim 1, where the fundus engagement device is covered with a cushion.

13. The massager of claim 1, where additional pressure is added to create a deeper massage.

14. The massager of claim 1, where the additional pressure may be selected from pneumatics and mechanical.

15. The massager of claim 1, where massager may be stored in a protective carrying case.

16. An automated fundal massager for massaging the uterus in a patient, the apparatus comprising:
a. at least one attachment strap;
b. a body housing the massager, power supply, and system controller;
c. at least one massager having—
i. at least one power supply,
ii. at least one roller pressingly engaging the fundus,
iii. at least one motor engaging the roller and maneuvering the roller throughout its range of motion, and
iv. at least one roller cover;
d. a system controller having—
i. at least one power control,
ii. at least one selectable mode control,
iii. at least one frequency settings control, and
iv. at least one speed control,
whereby the massager is placed and secured on a patient after delivery engaging the fundus of the uterus and providing a massage to maintain uterine firmness thereby managing postpartum bleeding.

17. The massager of claim 16, where the mode is selectable from continuous mode, ramping mode, and pulse mode.

18. The massager of claim 16, where the system controller is replaced by a microcontroller and programmable logic unit.

19. The massager of claim 16, where the controls are selected from and analog and digital controls.

20. The massager of claim 16, where the controls may controlled remotely by wired and wireless communication.
